(19) United States
(12) Patent Application Publication
(10) Pub. No.: US 2007/0179387 A1
(43) Pub. Date: Aug. 2, 2007

(54) Method and apparatus for measuring reserves of a periodically changing system

(76) Inventors: Horst Erhard Kunig, Saltsburg, PA (US); Anette Miriam Kunig, Saltsburg, PA (US)

Correspondence Address: Horst Erhard Kunig, P.O. Box 192, Saltsburg, PA, 15681-0192, US

(21) Appl. No.: 11/342,151

(22) Filed: Jan. 27, 2006

(51) Int. Cl. A61B 5/02

(52) U.S. Cl. 600/493

(57) ABSTRACT

A device is disclosed to determine functionality of the periodically changing system, to generate a functionality diagram and to measure and display functionality in said diagram. The device further measures efficiency and resource reserves of the system be comparison with efficiency and resource reference frames to determine deterioration and/or improvement of the system from the time changes of the reserves. The method and device have utility to design and monitor interventions for improvement of the system.

### FIELD OF THE INVENTION

The present invention relates to the functionality of a system periodically changing in time and, more specifically, to a method and apparatus for determining the reserves of the system before breakdown.

### DESCRIPTION OF PRIOR ART

For a periodically changing system functionality is usually inferred by relating maximal or minimal values of the periodically changing parameters during one cycle to an empirically derived surrogate range of normalcy. The empiricism associated with the surrogate range of normalcy provides for ambiguous functionality determinations. More specifically, absent are a lower limit minimal reference frame, below which the system does not function, and an upper limit maximal reference frame, above which the system also does not function. The difference between measured functionality and minimal and maximal reference frame is indicative of the reserve of the system.

Disclosed in U.S. Pat. No. 6,520,917 is a method to establish the synergy of several hemodynamic parameters from which to determine quantitatively functionality. This disclosure describes deterioration of the system by divergence from a minimal reference frame, but fails to delineate with specificity the conditions at which functionality ceases.

It is therefore an object of the present invention to provide minimal and maximal reference frames below and beyond which the system ceases to function.

It is a further objective of the present invention to compare measured functionality with minimal and maximal reference frames to allow determination of reserves.

It is a further objective of the present invention to determine deterioration and improvement from the change of the reserves in time.

It is still another object of the present invention to monitor functionality and to commence interventions upon approaching minimal and maximal reference frames to improve functionality and to monitor the benefits of the interventions.

### SUMMARY OF THE PRESENT INVENTION

According to the present invention, there is provided a device and a method for quantitative determination of the functionality of system. There is further provided a minimal and a maximal functionality reference frame for comparison with the measured functionality. Still further, there are provided means for determining the difference between measured functionality and minimal and maximal reference frame, said difference being used to determine the reserves the system. Further means are provided to determine deterioration from diminishing reserves over time and improvements from increasing reserves over time.

The device includes the combination of sensors responsive to parameters of a system periodically changing in time, collectively referred to as A, at an initial time t₁, denoted, A₁, and at a subsequent time t₂, denoted A₂, means to transmit A to a computer for computing the magnitudes of A at various times, the difference of the magnitudes of A at various times, the ratio of the change of A at various times in relation to the magnitude of A at an initial time, and the ratio of the change of A to the time in which the change occurred. The computer further includes sensors responsive to pre-selected minimal and maximal magnitudes of A, said minimal and maximal magnitudes comprising the minimal and maximal reference frames. The computer further includes means for comparison of instant functionality data with the minimal and maximal reference frames for determination of reserves and means for determining the change of the reserves with time and means for determining deterioration and improvement from said reserve changes in time. The device further provides means for determining the need for interventions and monitoring the effects of the interventions, depending on the changes of the reserves, and recording means, audible and visible warning means, activated upon the establishment of pre-selected values to warn of impending breakdown and modems for transmission to a central storage facility.

### BRIEF DESCRIPTION OF THE DRAWINGS

The present invention will be more fully understood when the following detailed description is read in conjunction with the accompanying drawings in which:

FIG. 1 illustrates the change of a parameter A with time t, said parameter A to include electrical, mechanical, electromechanical parameters, electrocardiographic signals, ECG, echocardiographic signals, ultrasound, arterial pressure, left ventricular pressure, atrial pressure, atrial volume, jugular pressure, central venous pressure, carotid pressure, radial pressure, pulmonary artery pressure, right ventricular pressure, ventricular volumes, ventricular cross-sectional areas, magnetic signals, bioimpedance signals, chemical signals, arterial oxygen concentration, venous oxygen concentration, oxygen consumption, temperature signals, time signals, frequency, heart rate, and combinations thereof, including but not limited to energies, and work, said signals collectively referred to as signals A;

FIG. 2 illustrates the utility of the instant invention in a functionality diagram to determine the functionality, functionality reserves, changes of the functionality reserves in time, and deterioration and improvement of the system;

FIG. 3 shows a block diagram of the apparatus to practice the instant invention;

FIG. 4 shows a functionality diagram of an exercising subject to determine deterioration and improvement;

FIG. 5 demonstrates the utility of determining exercising intensity levels from changing functionality reserves.

### DETAILED DESCRIPTION OF THE PREFERRED EMBODIMENT

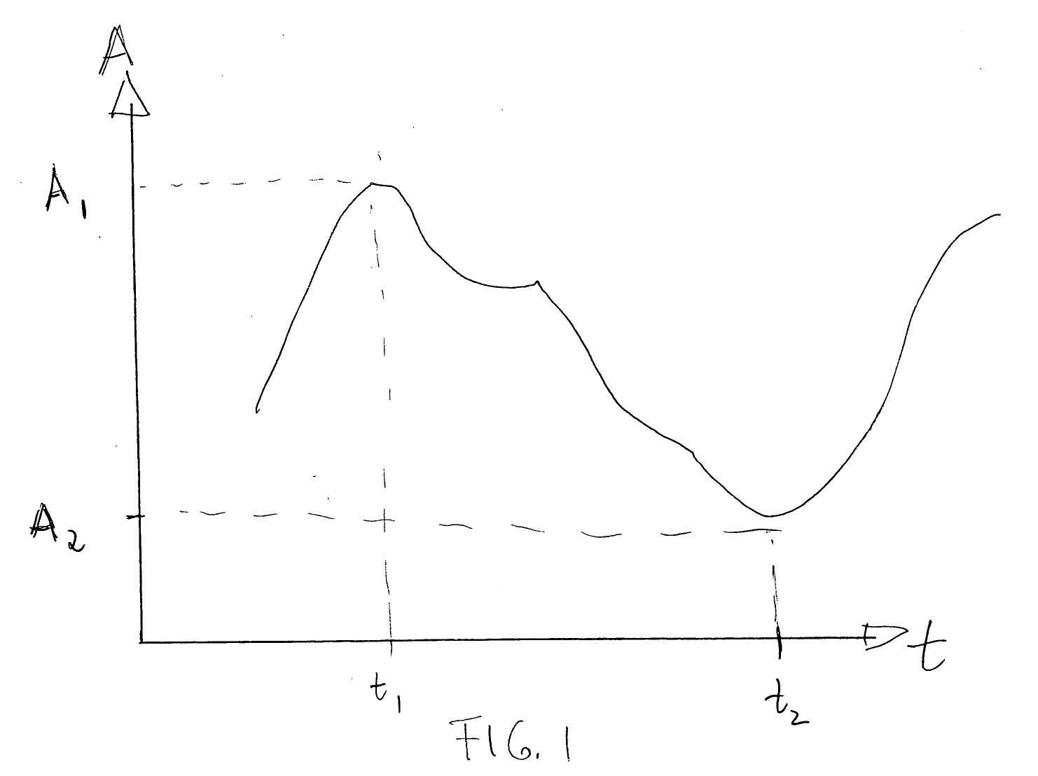

Referring now to FIG. 1, signals A are measured as a function of time, t. The magnitudes of signals A at specific times describe the state of the system at these instant times, thus, provide only snapshot information, but do not describe functionality. Functionality of the system is determined by the change of A, denoted AA, and given by AA=A₁−A₂, during the time t₂−t₁,
AA/(t₂−t₁)=(A₁−A₂)/(t₂−t₁) or
AA*=A₁*−A₂* (1)
Where AA*=AA/(t₂₋ₜ₁), A₁*=A₁/(t₂−t₁), and A₂*=A₂/(t₂−t₁). The frequency, f, of the periodically changing system, being equal to 1/((t₂−t₁)), may be substituted for the reciprocal of (t₂−t₁). Expanding the right side of equation (1) by the ratio of A₁*/A₁* yields
AA*=(A₁*−A₂*)×(A₁*/A₁*) or
AA*=EF(A)×A₁*, where (2)
EF(A)=(A₁*−A₂*)/A₁* (3)
is the ejection fraction. EF(A) denotes the efficiency of the system. Functionality equation (2) describes how efficiently (EF(A)) a resource (A₁*) is used by the system to function.

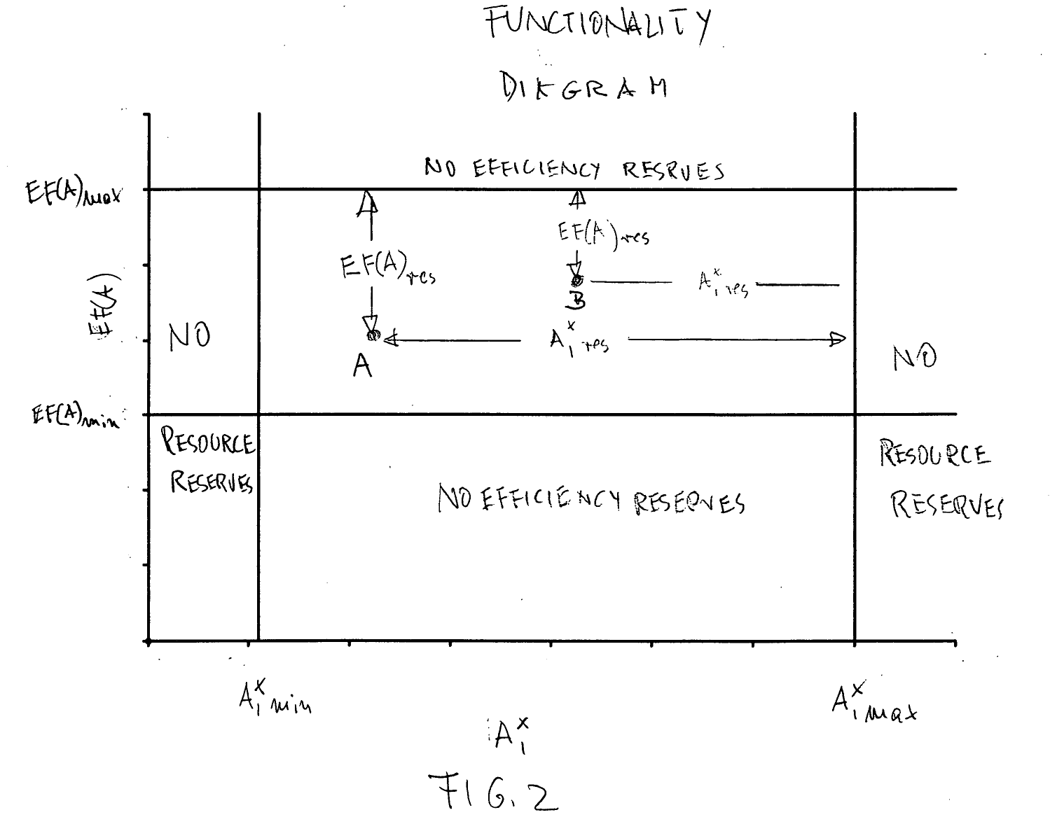

Referring now to FIG. 2, EF(A) is plotted versus A₁*, said plot being identified by the instant invention as functionality diagram. Minimal reference frames EF(A)ₘᵢₙ and A₁*ₘᵢₙ and maximal reference frames EF(A)ₘₐₓ and A₁*ₘₐₓ are added to the functionality diagram. The points in the functionality diagram having coordinates (A₁*ₘᵢₙ/EF(A)ₘᵢₙ), (A₁*ₘₐₓ/EF(A)ₘᵢₙ), (A₁*ₘₐₓ/EF(A)ₘₐₓ), and (A₁*ₘᵢₙ/EF(A)ₘₐₓ), delineate a rectangle. The system functions, if the measured values A₁* and EF(A) fall within the rectangle and ceases to function if the measured values A₁* and EF(A) fall outside the rectangle. More specifically, A₁*ₘᵢₙ<A₁<A₁*ₘₐₓ and EF(A)ₘᵢₙ<EF(A)<EF(A)ₘₐₓ denotes the area of functionality, and A₁*<A₁*ₘᵢₙ and A₁*>A₁*ₘₐₓ denote the area of dis-functionality and still further EF(A)<EF(A)ₘᵢₙ and EF(A)>EF(A)ₘₐₓ denote the area of dis-functionality. The difference A₁*ₘₐₓ and A₁* measures the resource reserves, A₁*ᵣₑₛ, and the difference EF(A)ₘᵢₙ and EF(A) measures the efficiency reserves, EF(A)ᵣₑₛ. Both reserves may be expressed as % of the maximal resources. Further the instant invention teaches, that diminishing reserves A₁*ᵣₑₛ and/or EF(A)ᵣₑₛ between two measurements denote deteriorating functionality, D, and increasing reserves A₁*ᵣₑₛ and/or EF(A)ᵣₑₛ between two measurements denote improved functionality, I. Also, as shown in FIG. 2, if A is a first measurement and B is a second measurement, then EF(A) and A₁*, diminishing between the two measurements, indicates deteriorating functionality, and vice versa.

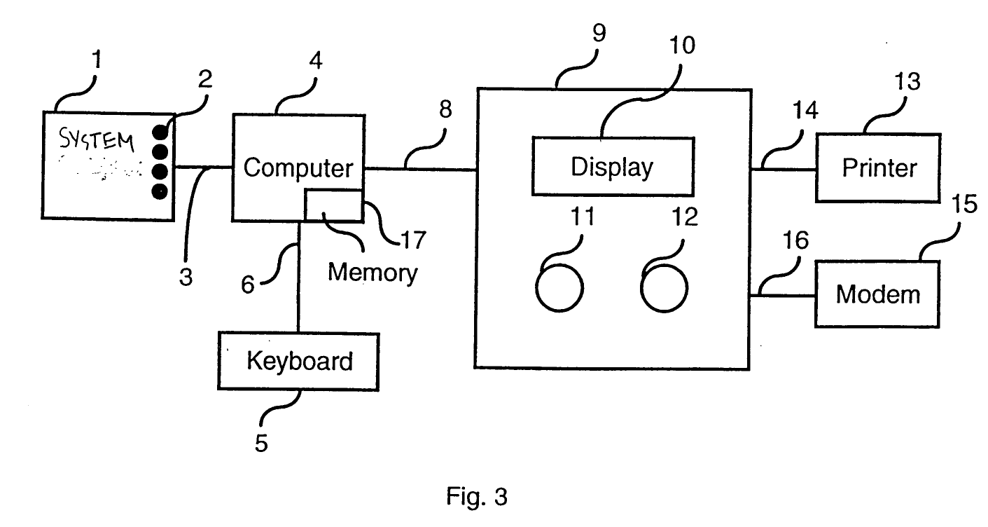

The embodiment, as shown in FIG. 3, illustrates the teachings of the instant invention. Accordingly, sensors 2 are placed on a system 1, said system to include the cardiocirculatory system to detect signals representative of signals A to include but not limited to electrical, mechanical, electromechanical parameters, electrocardiographic signals, ECG, echocardiographic signals, ultrasound, arterial pressure, left ventricular pressure, atrial pressure, atrial volume, jugular pressure, central venous pressure, carotid pressure, radial pressure, pulmonary artery pressure, right ventricular pressure, ventricular volumes, ventricular cross-sectional areas, magnetic signals, bioimpedance signals, chemical signals, arterial oxygen concentration, venous oxygen concentration, oxygen consumption, temperature signals, time signals, frequency, heart rate, and combinations thereof, including but not limited to energies, and work, said signals collectively referred to as signals A, which are transmitted on multi-line wire 3 to computer 4. Such sensors 2 may include catheters, electrodes, electrocardiographs, bioimpedance measuring equipment magnetic resonance measuring equipment, ultra-sound equipment, pressure transducers, pressure cuffs, temperature sensors, chemical sensors, time sensors, and echocardiographic sensors. Additional input representative of patient information including weight, height, body surface area, pre-selected time intervals, and pre-selected minimal and maximal reference frames is provided from a keyboard 5 to computer 4 on line 6. Computer 4 is programmed to process the incoming signals on line 6 to establish reference frames A₁*ₘᵢₙ, A₁*ₘₐₓ, EF(A)ₘᵢₙ, and EF(A)ₘₐₓ for determining zones of functionality and dis-functionality. Computer 4 is also programmed to process the incoming signals on line 3, to determine their magnitudes, the changes of the magnitudes in relation to an initial magnitude and to the time in which the changes occurred to construct a functionality diagram, and to compare measured data EF(A) and A₁*, entered in the functionality diagram with minimal and maximal reference frames A₁*ₘᵢₙ, A₁*ₘₐₓ, EF(A)ₘᵢₙ, and EF(A)ₘₐₓ, for further determination of efficiency reserves (EF(A)ᵣₑₛ and resource reserves A₁*ᵣₑₛ. Computer 4 also determines the need for interventions upon attainment of pre-determined values of resource reserves A₁*ᵣₑₛ, pre-determined values of efficiency reserves EF(A)ᵣₑₛ and to monitor the benefits of interventions by monitoring the changes of A₁*ᵣₑₛ and EF(A)ᵣₑₛ caused by the intervention, indicative of deteriorating or improving functionality. All parameters, representative of said functionality, are transmitted by line 8 to a monitor 9 which is comprised of a display 10, audible and visual alarms 11 to warn of emergencies if preset values of the parameters are attained, and indicators 12 to display the functionality diagram, minimal and maximal reference frames, resource and efficiency reserves, and deteriorating and improving functionality. The signals displayed by display 10 and the audio and visual alarms 11 and the signals displayed by indicator 12 are transmitted on line 14 to a printer 13 for producing hard copies and on line 16 to a modem 15 for transmission to central storage and retrieval. A memory 17 in the computer 4 serves as storage of all information and data.

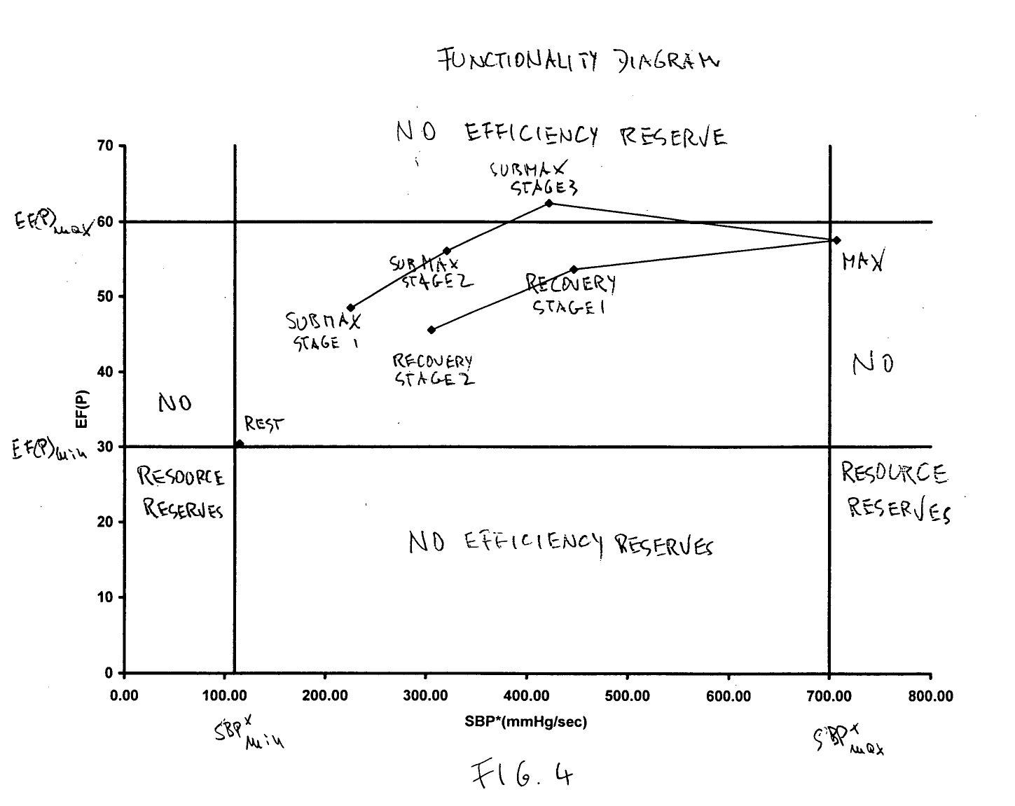

Referring now to FIG. 4, there is displayed a functionality diagram generated from data as published by R. A. Wolthuis et al. in an article, entitled, The response of health men to treadmill exercise, Circulation 1977; 55:153-157, which are summarized in Table 1. Here the system is comprised of the cardiocirculatory system, the signal A is the arterial blood pressure, A₁ is the systolic blood pressure SBP, A₂ is the diastolic pressure, DBP, and the frequency f is the heart rate, HR. Measurements, displayed in the functionality diagram, were taken at rest, at three sub-maximal exercise stages of increasing intensities, at maximal intensity, and at two subsequent times during recovery. Maximal reference frames SBP*ₘₐₓ of 700 mm Hg/sec and EF(P)ₘₐₓ of 60% were derived from the data at maximal exercise intensities SBP of 212 mm Hg, DBP of 75 mm Hg, and heart rate of 220 mm Hg. Minimal reference frames SBP*ₘᵢₙ of 115 mm Hg/sec and EF(P)ₘᵢₙ of 30% were generated from data at rest for subjects of the age group of 20 years to 30 years, as published in Ciba-Geigy Scientific Tables, Ciba-Geigy Corporation, Medical Education Division, West Caldwell, N.J. 07006, ISBN 0-914168-54-1, 1990 of SBP equal to 115 mm Hg, DBP equal to 80 mm Hg, and HR equal to 60 l/min. As shown in Table 1, EF(A)ᵣₑₛ and SBP*ᵣₑₛ continuously decrease with increasing exercise intensity until, maximal efforts are expended, indicating deterioration, and continuously increase during the recovery period, indicating improvement. In this embodiment the present invention teaches the determination of the instant cardiocirculatory reserves non-invasively without the involvement of skilled personnel and, further, the need for intervention or discontinuation of an exercising activity at pre-determined values of the reserves. Further, according to the instant inventions, athletes, desiring to improve their competitiveness and subjects desiring to maintain and improve their fitness can select the proper exercise intensity level, given by the maximal efficiency reference frame, instead of relying, as presently, on individual statistically derived parameters, such as age-dependent target heart rate, given by the empirical formula 220 minus age.

TABLE 1

| | | | |
|---|---|---|---|
| SBP | 115 | mmHg | rest |
| SBP | 132 | mmHg | sub-maximal stage 1 |
| SBP | 148 | mmHg | sub-maximal stage 2 |
| SBP | 160 | mmHg | sub-maximal stage 3 |
| SBP | 212 | mmHg | maximal |
| SBP | 194 | mmHg | recovery stage 1 |
| SBP | 158 | mmHg | recovery stage 2 |
| DBP | 80 | mmHg | rest |
| DBP | 68 | mmHg | sub-maximal stage 1 |
| DBP | 65 | mmHg | sub-maximal stage 2 |
| DBP | 60 | mmHg | sub-maximal stage 3 |
| DBP | 75 | mmHg | maximal |
| DBP | 90 | mmHg | recovery stage 1 |
| DBP | 86 | mmHg | recovery stage 2 |
| HR | 60 | 1/min | rest |
| HR | 102 | 1/min | sub-maximal stage 1 |
| HR | 130 | 1/min | sub-maximal stage 2 |
| HR | 158 | 1/min | sub-maximal stage 3 |
| HR | 200 | 1/min | maximal |
| HR | 138 | 1/min | recovery stage 1 |
| HR | 116 | mmHg | recovery stage 2 |

| | | |
|---|---|---|
| EF(P) | 30% | rest |
| EF(P) | 48% | sub-maximal stage 1 |
| EF(P) | 56% | sub-maximal stage 2 |
| EF(P) | 63% | sub-maximal stage 3 |
| EF(P) | 60% | maximal |
| EF(P) | 54% | recovery stage 1 |
| EF(P) | 46% | recovery stage 2 |
| EF(P)ᵣₑₛ | 50% | rest |
| EF(P)ᵣₑₛ | 19% | sub-maximal stage 1 |
| EF(P)ᵣₑₛ | 7% | sub-maximal stage 2 |
| EF(P)ᵣₑₛ | 0% | sub-maximal stage 3 |
| EF(P)ᵣₑₛ | 4% | maximal |
| EF(P)ᵣₑₛ | 11% | recovery stage 1 |
| EF(P)ᵣₑₛ | 24% | recovery stage 2 |
| SBP*ᵣₑₛ | 84% | rest |
| SBP*ᵣₑₛ | 63% | sub-maximal stage 1 |
| SBP*ᵣₑₛ | 54% | sub-maximal stage 2 |
| SBP*ᵣₑₛ | 40% | sub-maximal stage 3 |
| SBP*ᵣₑₛ | 0% | maximal |
| SBP*ᵣₑₛ | 36% | recovery stage 1 |
| SBP*ᵣₑₛ | 56% | recovery stage 2 |
| EF(P)ₘᵢₙ | 30% | |
| EF(P)ₘₐₓ | 60% | |

| | | |
|---|---|---|
| SBP*ₘᵢₙ | 115 | mmHg/sec |
| SBP*ₘₐₓ | 700 | mmHg/sec |

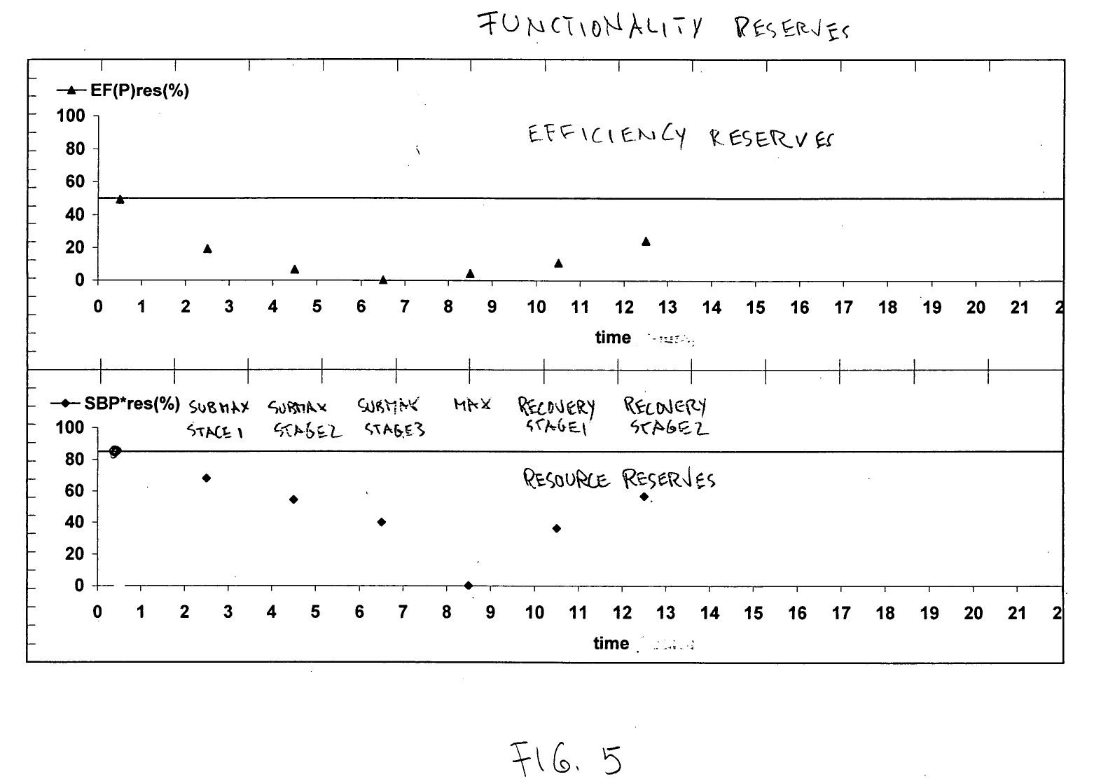

Referring now to FIG. 5, there is displayed a time resolution of efficiency EF(P)ᵣₑₛ and resource SBP*ᵣₑₛ reserves in two separate graphs, said graphs being derived from the exercising subject, whose functionality diagram is shown in FIG. 4. This display has added utility of delineating the time at which specific magnitudes of EF(P) and SBP* occur, which otherwise is embedded in the three dimensional FIG. 4. Still further, the instant invention allows determination of efficiency and resource reserves at any given time without the need for stressing the system to near breakdown conditions.

In other embodiments of the present invention other parameters including but not limited to electrical, mechanical, electromechanical parameters, electrocardiographic signals, ECG, echocardiographic signals, ultrasound, arterial pressure, left ventricular pressure, atrial pressure, atrial volume, jugular pressure, central venous pressure, carotid pressure, radial pressure, pulmonary artery pressure, right ventricular pressure, ventricular volumes, ventricular cross-sectional areas, magnetic signals, bioimpedance signals, chemical signals, arterial oxygen concentration, venous oxygen concentration, oxygen consumption, body surface area, body mass index, temperature signals, time signals, frequency, heart rate, and combinations thereof, including but not limited to energies, and work, together with other constant parameters to serve as reference frames said parameters to be used to determine functionality to be further used to select interventions and to monitor improvement and/or deterioration, and to evaluate the benefits of the interventions.

While the present invention has been described in connection with the preferred embodiments of the various figures, it is to be understood that other similar embodiments may be used or modifications and additions may be made to the described embodiment for performing the same functions of the present invention without deviating therefrom. Therefore, the present invention should not be limited to any single embodiment, but rather construed in breadth and scope in accordance with the recitation of the appended claims.

We claim:

1. A device for establishing functionality of a system consisting of:
means responsive for measuring parameters of a system;
means responsive to the measurements of parameters;
means for providing functionality equations;
means for computing to derive functionality from measured parameters and functionality equations;
means for deriving a functionality in a functionality diagram;
means for establishing zones of functionality and dis-functionality in the functionality diagram;
means for measuring functionality in the functionality diagram;
means for measuring reserves and deterioration and improvement in the functionality diagram;
means for display of functionality, reserves, deterioration, and improvement in a functionality diagram.

2. The cardiac diagnostic device according to claim 1 wherein said measurements of parameters include signals changing in time.

3. The device according to claim 1 wherein said means for deriving said functionality diagram includes a computer for establishing minimal and maximal reference frames for said functionality diagram from inputs of multiples of constant parameters via a keyboard, said reference frames are used to further establish zones of functionality and dis-functionality.

4. The device according to claim 3 wherein said means for establishing functionality includes said computer for determining functionality from the functionality equations
AA*=EF(A)×A₁*
AA*=A₁*−A₂*
EF(A)=(A₁−A₂)/A₁
wherein AA*, A₁*, and A₂* equal AA, A₁, and A₂ referenced to time and reference frames EF(A)ₘᵢₙ, EF(A)ₘₐₓ, A₁*ₘᵢₙ, and A₁*ₘₐₓ, and wherein A₁ is a parameter, measured at time t₁, A₂ is a parameter, measured at time t₂, and AA is the difference of A₁ and A₂.

5. The device according to claim 4 wherein said computer measures functionality within the reference frames of the functionality diagram for EF(A)ₘᵢₙ<EF(A)<EF(A)ₘₐₓ and A₁*ₘᵢₙ<A₁*<A₁*ₘₐₓ and dis-functionality for EF(A)<EF(A)ₘᵢₙ and EF(A)>EF(A)ₘₐₓ indicating lack of efficiency reserves, and dis-functionality for A₁*<A₁*ₘᵢₙ and A₁*>A₁*ₘₐₓ, indicating lack of resource reserves.

6. The device according to claim 5 wherein said computer determines efficiency reserves, EF(A)ᵣₑₛ, of the system from the difference of EF(A)ₘₐₓ and EF(A) and resource reserves, A₁*ᵣₑₛ, from difference of A₁*ₘₐₓ and A₁*, and deterioration, when efficiency reserves and/or resource reserves decline over time, and improvement, when efficiency reserves and/or resource reserves increase over time.

7. The device according to claim 6 to design and monitor system-specific interventions for improvement of efficiency and resource reserves.

8. The device according to claim 6 to determine cardiocirculatory fitness and to design and monitor patient-specific rehabilitation and subject-specific conditioning programs.

9. The device of claim 6 wherein said computer evaluates the efficacy of drugs by analyzing efficiency and resource reserves to effectuate deterioration and improvement in patients.

10. The device according to claim 1 wherein said parameters include electrical, mechanical, electromechanical parameters, electrocardiographic signals, ECG, echocardiographic signals, ultrasound, arterial pressure, left ventricular pressure, atrial pressure, atrial volume, jugular pressure, central venous pressure, carotid pressure, radial pressure, pulmonary artery pressure, right ventricular pressure, ventricular volumes, ventricular cross-sectional areas, magnetic signals, bioimpedance signals, chemical signals, arterial oxygen concentration, venous oxygen concentration, oxygen consumption, temperature signals, time signals, frequency, heart rate, and combinations thereof, including but not limited to energies, and work.

11. The device of claim 6 wherein said means responsive to the measurement of said signals include catheters, electrodes, electrocardiographs, bioimpedance measuring equipment magnetic resonance measuring equipment, ultra-sound equipment, pressure transducers, pressure cuffs, temperature sensors, chemical sensors, time sensors, and echocardiographic sensors and additional means responsive to input representative of patient information including weight, height, body surface area, pre-selected time intervals, and pre-selected minimal and maximal reference frames.

12. A method of diagnosing functionality of a system; said method including the steps of:
measuring parameters A of said system at an initial time t₁, denoted A₁, and at a subsequent time t₂, denoted A₂;
establishing functionality from the functionality equations
AA*=EF(A)×A₁*
AA*=A₁*×A₂*
EF(A)=(A₁−A₂)/A₁)
wherein performance data AA*, A₁*, and A₂* equal measured data AA, A₁, and A₂ referenced to time,
establishing a functionality diagram;
establishing maximal and minimal reference frames in the functionality diagram;
comparing measured and derived performance data to the reference frames for computing efficiency and resource reserves, determining deterioration and improvement from the time changes of declining or increasing reserves and display of said data in the functionality diagram;

13. The method of claim 12 including the steps of design and monitoring of system-specific interventions for improvement of the reserves.

14. The method of claim 12 including the steps of design and monitoring patient-specific rehabilitation and subject-specific conditioning programs.

15. The method of claim 12 including the steps of evaluating the efficacy of drugs in a functionality diagram.

16. The method of claim 12 wherein said step of measuring includes parameters changing in time, electrical, mechanical, electromechanical parameters, electrocardiographic signals, ECG, echocardiographic signals, ultrasound, arterial pressure, left ventricular pressure, atrial pressure, atrial volume, jugular pressure, central venous pressure, carotid pressure, radial pressure, pulmonary artery pressure, right ventricular pressure, ventricular volumes, ventricular cross-sectional areas, magnetic signals, bioimpedance signals, chemical signals, arterial oxygen concentration, venous oxygen concentration, oxygen consumption, temperature signals, time signals, frequency, heart rate, and combinations thereof, including but not limited to energies, and work

17. The method of claim 12 wherein said means responsive to the measurement of said signals include catheters, electrodes, electrocardiographs, bioimpedance measuring equipment magnetic resonance measuring equipment, ultra-sound equipment, pressure transducers, pressure cuffs, temperature sensors, chemical sensors, time sensors, and echocardiographic sensors and additional means responsive to input representative of patient information including weight, height, body surface area, pre-selected time intervals, and pre-selected minimal and maximal reference frames.
